(19) United States
(12) Patent Application Publication
(10) Pub. No.: US 2013/0345526 A1
(43) Pub. Date: Dec. 26, 2013

(54) Automated Prevention and Control of Epileptic Seizures Using Biochemical And/or Electrical Signal Markers

(71) Applicant: FLINT HILLS SCIENTIFIC, LLC, Lawrence, KS (US)

(72) Inventor: Ivan Osorio, Leawood, KS (US)

(73) Assignee: FLINT HILLS SCIENTIFIC, LLC, Lawrence, KS (US)

(21) Appl. No.: 13/789,320

(22) Filed: Mar. 7, 2013

Related U.S. Application Data
(60) Provisional application No. 61/664,071, filed on Jun. 25, 2012.

(51) Int. Cl. A61B 5/00; A61N 1/00; A61B 5/0476; A61B 5/145

(52) U.S. Cl. CPC A61B 5/4094; A61B 5/14546; A61B 5/7275; A61B 5/7282; A61B 5/4839; A61B 5/14503; A61N 1/00; A61B 5/0476
USPC 600/309

(57) ABSTRACT

Methods and medical device systems for preventing or abating seizures, making use of an association between the probability or likelihood of seizure occurrence and the concentrations or activity levels of one or more of putrescine, spermidine, spermine, or γγ-aminobutyric acid (GABA). The concentration or activity level of putrescine, spermidine, spermine, or GABA, or a substrate or enzyme for synthesis thereof, may be determined, and at least one further action may be performed.

The present application claims the benefit of prior-filed, copending U.S. Provisional Patent Appln. 61/664,071, filed Jun. 25, 2012, which is hereby incorporated herein by reference.

### FIELD OF THE INVENTION

This disclosure relates to medical device systems and methods capable of preventing and/or abating epileptic seizures.

### SUMMARY OF THE INVENTION

In some embodiments, the present disclosure relates to a method, comprising: determining a concentration of at least one polyamine selected from putrescine, spermidine, spermine, or γ-aminobutyric acid (GABA) in a brain of a patient; and taking a further action selected from: issuing a warning if the concentration is below a first reference value; indicating a lower probability of an epileptic event if the concentration is above a second reference value; increasing the concentration if the concentration is below a third reference value; or maintaining the concentration above a fourth reference value.

In some embodiments, the present disclosure relates to a method, comprising: determining an occurrence of a seizure in a patient; and increasing a concentration of at least one polyamine selected from putrescine, spermidine, spermine, or γ-aminobutyric acid (GABA) in a brain of the patient in response to the determining, within a predetermined time period after the occurrence of the seizure.

In some embodiments, the present disclosure relates to a method, comprising: determining, at a first time point, a first concentration or an activity level of at least one of: a polyamine selected from putrescine, spermidine, spermine, or γ-aminobutyric acid (GABA); a substrate for synthesis of putrescine, spermidine, spermine, or GABA; or an enzyme for synthesis of putrescine, spermidine, spermine, or GABA; in a brain of a patient; estimating a second concentration of at least one polyamine, expected at a second, future time point; and taking a further action selected from: issuing a warning if the second concentration is expected to be below a first reference value; indicating a lower probability of an epileptic event if the second concentration is expected to be above a second reference value; increasing the first concentration if the second concentration is expected to be below a third reference value; or maintaining the first concentration above a fourth reference value.

In some embodiments, the present disclosure relates to a method, comprising: determining a concentration of at least one polyamine selected from putrescine, spermidine, spermine, or γ-aminobutyric acid (GABA) in a brain of a patient; determining a probability of an occurrence of at least one seizure during a first time window, based on the concentration; and taking a further action selected from: issuing a warning if the probability is above a first reference value; indicating a lower probability of an epileptic event if the probability is below a second reference value; increasing the concentration if the probability is above a third reference value; or maintaining the concentration if the probability is below a fourth reference value.

In some embodiments, the present disclosure relates to a method, comprising determining a concentration of a substrate for synthesis of putrescine, spermidine, spermine, or γ-aminobutyric acid (GABA), a concentration or an activity level of an enzyme for synthesis of putrescine, spermidine, spermine, or GABA, or a concentration of a metabolite of one of putrescine, spermidine, spermine, or GABA in a brain of a patient; and taking a further action selected from: issuing a warning if the concentration is below a first reference value; indicating a lower probability of an epileptic event if the concentration is above a second reference value; increasing the concentration if the concentration is below a third reference value; or maintaining the concentration above a fourth reference value.

In some embodiments, the present disclosure relates to a medical device system, comprising (i) at least one sensor configured to collect data relating to at least one of: a concentration of a polyamine selected from putrescine, spermidine, spermine, or γ-aminobutyric acid (GABA); a concentration of a substrate for synthesis of putrescine, spermidine, spermine, or GABA; or a concentration or level of activity of an enzyme for synthesis of putrescine, spermidine, spermine, or GABA in a brain of a patient; (ii) at least one processor configured to: (ii-a) determine the concentration of a polyamine, the concentration of a substrate for synthesis of a polyamine, or the concentration or level of activity of an enzyme for synthesis of a polyamine from the collected data; (ii-b) perform one or more of: (ii-b-1) estimate a future concentration of a polyamine, the substrate for synthesis of a polyamine, or the concentration or level of activity of an enzyme for synthesis of a polyamine; (ii-b-2) compare the concentration to a reference value; or (ii-b-3) determine, as function of the concentration or level of activity, a probability of an occurrence of at least one seizure during a first time window; and (ii-c) issue a signal relating to a further action, based on at least one of the estimated future concentration or level of activity, the compared or level of activity concentration, or the determined probability; and at least one of: (iii) a warning unit configured to provide a warning relating to at least one of the estimated future concentration or activity level, the compared concentration or activity level, or the determined probability; (iv) a concentration/activity level increasing unit configured to increase the concentration/activity level, based on at least one of the estimated future concentration/activity level, the compared concentration/activity level, or the determined probability; (v) a probability indication unit configured to indicate a lower probability of an epileptic event, based on at least one of the estimated future concentration/activity level, the compared concentration/activity level, or the determined probability; or (vi) a concentration/activity level maintaining unit configured to maintain the concentration/activity level, based on at least one of the estimated future concentration/activity level, the compared concentration/activity level, or the determined probability.

In some embodiments, the present disclosure relates to a medical device system, comprising (i) at least one sensor configured to collect data relating to at least one of: a concentration of a polyamine selected from putrescine, spermidine, spermine, or γ-aminobutyric acid (GABA); a concentration of a substrate for synthesis of putrescine, spermidine, spermine, or GABA or a metabolite thereof; or a concentration/activity level of an enzyme for synthesis of putrescine, spermidine, spermine, or GABA in a brain of a patient; and (ii) at least one processor configured to: determine the concentration of a polyamine, the concentration of a substrate for synthesis of a polyamine, the concentration or the activity level of an enzyme for synthesis of a polyamine, or the concentration of a metabolite of a polyamine; determine a seizure susceptibility index relating to a probability of an occurrence of a seizure; and perform at least one of: issue a warning if the seizure susceptibility index is above a first reference value; indicate a lower probability of an epileptic event if the seizure susceptibility index is below a second reference value; increase the concentration if the seizure susceptibility index is above a third reference value; or maintain the concentration if the seizure susceptibility index is below a fourth reference value.

In some embodiments, the present disclosure relates to a method, comprising recording electrical brain activity, determining a concentration of at least one polyamine selected from putrescine, spermidine, spermine, or γ-aminobutyric acid (GABA) or a metabolite thereof; a concentration of a substrate for synthesis of putrescine, spermidine, spermine, or GABA; or a concentration/activity level of an enzyme for synthesis of putrescine, spermidine, spermine, or GABA in a brain of a patient in response to a change in the electrical activity, wherein the change is indicative of an increase in a seizure susceptibility index, and taking a further action selected from: issuing a warning if the change in electrical activity is associated with a decrease in concentration of a polyamine, initiating at least one action to increase the concentration in response to the seizure susceptibility index being above a first reference value; indicating a lower probability of an epileptic event if the seizure susceptibility index is below a second reference value; or maintaining the concentration above a third reference value.

In some embodiments, the present disclosure relates to a method, comprising determining a concentration of at least one polyamine selected from putrescine, spermidine, spermine, or γ-aminobutyric acid (GABA), or a metabolite, substrate, or enzyme for synthesis thereof (or activity of the enzyme) in a brain of a patient in response to at least one of time of day, state of the epileptogenic network, state of the brain, state of the patient, or in response to changes in the environment, and taking a further action selected from: issuing a warning if the concentration is below a first reference value; indicating a lower probability of an epileptic event if the concentration is above a second reference value; increasing the concentration if the concentration is below a third reference value; or maintaining the concentration above a fourth reference value.

In some embodiments, the present disclosure relates to a non-transitory computer readable program storage unit encoded with instructions that, when executed by a computer, perform a method as described above.

### BRIEF DESCRIPTION OF THE DRAWINGS

The disclosure may be understood by reference to the following description taken in conjunction with the accompanying drawings, in which like reference numerals identify like elements, and in which:

FIG. 1A shows the empirical probability (0-70%; y-axis) of being in seizure as a function of time (x-axis) elapsed before onset and after termination of a seizure, in human patients.

FIG. 1B shows the increase in seizure likelihood (from 0.9x to about 1.45x baseline; y-axis) as a function of time elapsed (x-axis) before onset and after termination of a seizure, in an animal model.

FIG. 2A shows the time to next seizure (y-axis) increases as a function of time elapsed since the last (previous) seizure (x-axis), in human patients.

FIG. 2B shows the time to next seizure (y-axis) increases as a function of time elapsed since the last (previous) seizure (x-axis), in an animal model.

FIG. 3A shows the biosynthesis and interconversion pathways of polyamines.

FIG. 3B shows pathways of GABA formation.

FIG. 4 shows biosynthetic and catabolic reactions of the polyamines.

FIG. 5 summarizes the pathways of biosynthesis and interconversion of polyamines.

FIG. 6 shows the role of antizyme and ODC in polyamine metabolism.

FIG. 7 shows pathways of 4-aminobutyraldehyde formation from putrescine, as well as cytotoxic compounds generated as by-products of the depicted reactions. DAO=diamine oxidase.

FIG. 8 shows the polyamine metabolic pathway, including indications of which reactions are spontaneous (open arrows) or enzyme driven (filled arrows).

FIG. 9 shows a model of polyamine circulation. Solid black bar, NMDA receptor; solid white bar without arrow, AMPA/Kainate receptor; solid gray bar, Kir channel; solid white bar with arrow, membrane-potential dependent transporter; hatched box with arrow, proton-gradient dependent transporter; large circle, synaptic vesicle; small circle, polyamine

FIG. 10 presents a block diagram of a medical device system, in accordance with some embodiments of the present disclosure.

FIGS. 11-16 present flowcharts of a number of methods, in accordance with some embodiments of the present disclosure.

The disclosure is susceptible to various modifications and alternative forms. Specific embodiments are shown by way of example in the drawings and are herein described in detail. However, the description of specific embodiments is not intended to limit the disclosure to the particular forms disclosed. The present disclosure covers all modifications, equivalents, and alternatives falling within the spirit and scope of the invention as defined by the appended claims.

### DETAILED DESCRIPTION OF SPECIFIC EMBODIMENTS

Illustrative embodiments of the disclosure are described herein. For clarity, not all features of an actual implementation are described. In the development of any actual embodiment, numerous implementation-specific decisions must be made to achieve design-specific goals, which will vary from one implementation to another. Such a development effort, while possibly complex and time-consuming, would nevertheless be a routine undertaking for persons of ordinary skill in the art having the benefit of this disclosure.

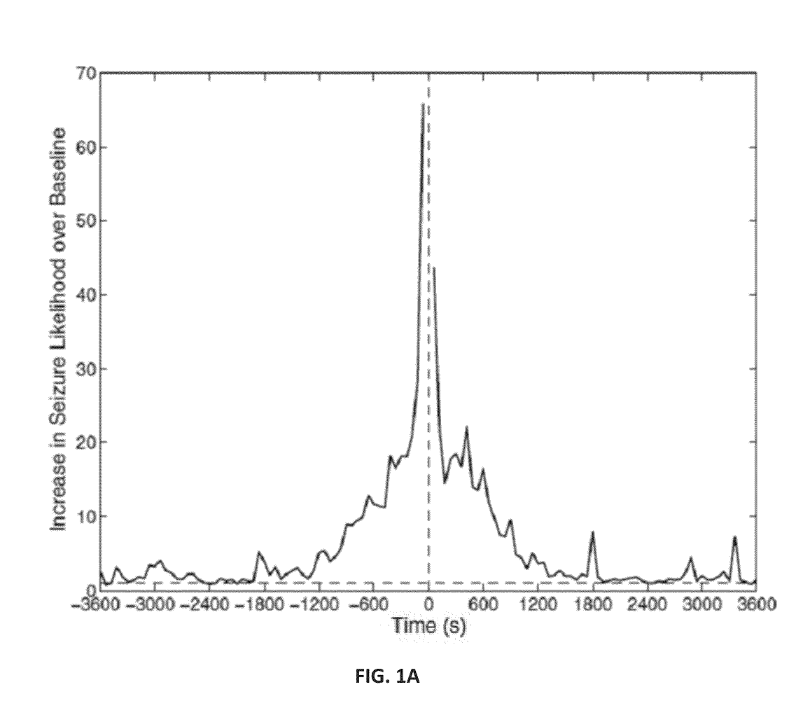

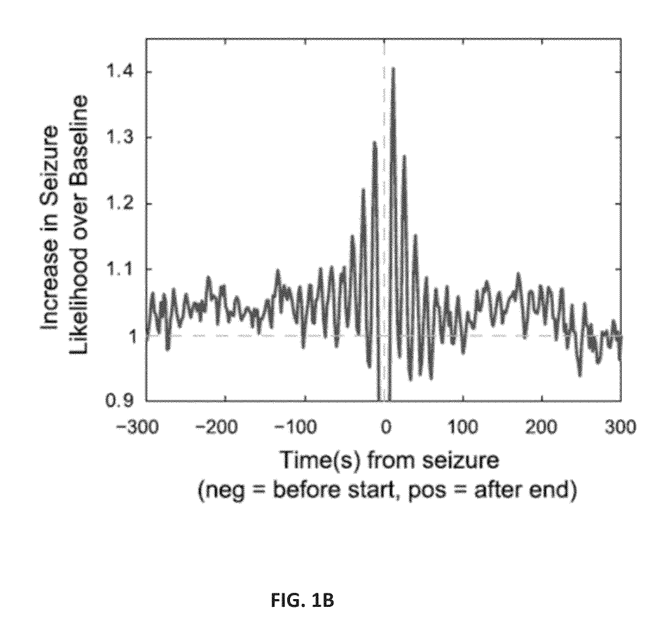

FIG. 1A shows the empirical increase in probability relative to baseline (y-axis) of about 60 human subjects being in a seizure state as a function of time elapsed (x-axis) before onset and after termination of a seizure. The curve to the left of the vertical dashed line (at time zero) depicts the probability before onset and the one to the right of this line depicts the probability after seizure termination. The empirical probability of being in seizure increases approximately 1800 sec before onset and returns to baseline about 1800 sec after termination of a given seizure, with the most pronounced increase occurring about 1200 sec before onset and ending about 1200 sec after termination. This behavior is indicative of a strong clustering tendency (“seizures beget seizures”), explaining why the conditional probability of seizure occurrence in subjects with pharmaco-resistant epilepsy is highest before onset and after termination of a seizure, with the period of increased probability spanning a total of about 60 minutes, approximately equally divided between the pre-and post-ictal periods.

This window of increased ictal susceptibility revealed by FIG. 1A for human patients was also found in a rat model of generalized seizures, albeit with much shorter duration (from approximately 100 sec before onset to about 100 sec after termination), as shown in FIG. 1B.

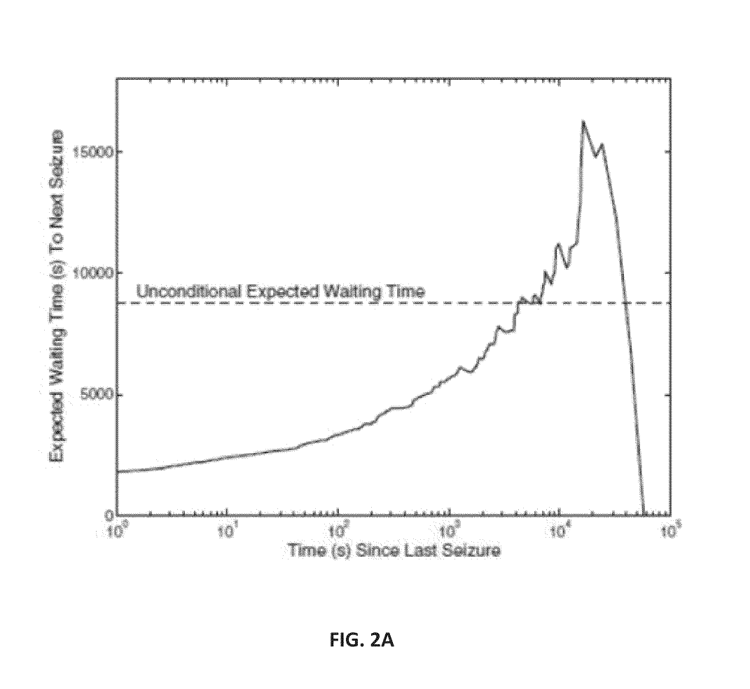

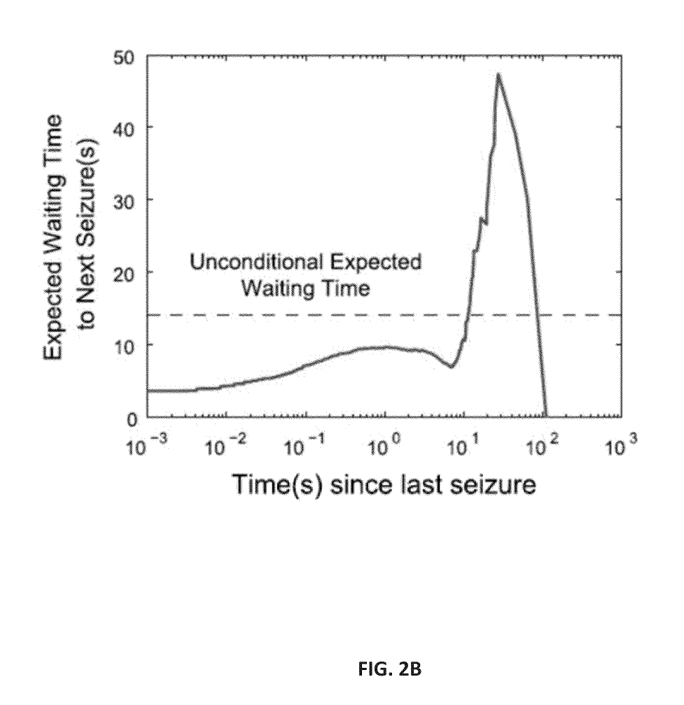

Related to this, FIGS. 2A-B summarize observations in humans (FIG. 2A) and rats (FIG. 2B) that the longer the time elapsed since the last seizure, the longer the time until the next one. It should be noted that the sharp decreases in expected waiting time after about 20,000 sec in humans (FIG. 2A) and after about 30 sec in rats (FIG. 2B) arose from the close of the observing window in each experiment. To construct the curves of FIGS. 2A and 2B, if the system was waiting for a next seizure at the closing of the observing window, the observed waiting time was treated as the actual waiting time, even though no next seizure had actually occurred. Therefore, the sharp decreases do not represent an actual decrease in expected waiting time for seizures of the listed durations).

FIGS. 2A-2B indicate that a transient increase in susceptibility to seizure generation and/or occurrence may occur in close temporal proximity to seizure onset and termination, that is followed by a more sustained decrease in susceptibility/probability of occurrence that is correlated with time (e.g., the longer the time elapsed since the last seizure, the lower the probability of seizure occurrence). This bi-phasic behavior (increased probability for a short time after a seizure and a “monotonic” decrease in probability after a certain time), manifesting in temporally closely spaced (cluster) seizures and long intervals of freedom from seizures (FIGS. 1, 2A-B), may be exploited for treatment purposes. The transient, short-lived (compared to the length of the period of decreased susceptibility) increase in seizure proclivity after the termination of a seizure suggests the epileptogenic network in the short window of time following a seizure is close to a critical threshold that facilitates inter-ictal to ictal transitions. During this period of increase susceptibility immediately following a seizure, dynamical (relating to brain activity) or extraneous “noise” (relating to stimuli such as lights) that would not trigger a seizure at times remote from a seizure may trigger a seizure given the proximity of the epileptogenic network to the seizure threshold.

More recent observations on Xenopus tadpoles treated with pentylenetetrazol, a seizure-inducing compound, lend support to those made in humans and rodents discussed above. A “priming” seizure significantly delayed onset of subsequent seizure in tadpoles compared to the unprimed controls, but only after approximately 2 hr had elapsed from the termination of the “priming” seizure; in the 2 hrs immediately following the “priming” seizure, the probability of emergence of seizures remained high. The decreased susceptibility to seizure emergence in amphibians 2 hrs after seizure termination was attributed to elevations in the concentration in brain of the diamine putrescine (NH₂(CH₂)₄NH₂). An increase in the concentration of this compound was temporally correlated with the decreased probability of seizure occurrence in these amphibians.

Putrescine and the related polyamines spermidine (NH₂(CH₂)₄NH(CH₂)₃NH₂) and spermine (NH₂(CH₂)₃NH(CH₂)₄NH(CH₂)₃NH₂) are biological cations, given that all amino groups are protonated at physiological pH. These polyamines have been implicated in many physiological processes, such as 1. Cell growth and differentiation; 2. Regulation of ion transport across membrane channels (e.g., inwardly rectifying K⁺ current); 3. Activation or blockage of NMDA receptors; 4. Blockage of AMPA or kainite receptors; and, 5. Cellular apoptosis.

Putrescine, spermidine, spermine, and γ-aminobutyric acid (GABA) may together be referred to herein as “polyamines.” Putrescine is included under this term for simplicity, even though it is a diamine, and GABA is also included for simplicity, even though it is a monoamine. Persons of ordinary skill in the art will understand from the context herein whether “polyamine” refers to putrescine, spermidine, spermine, and GABA on the one hand, or compounds comprising three or more amines on the other.

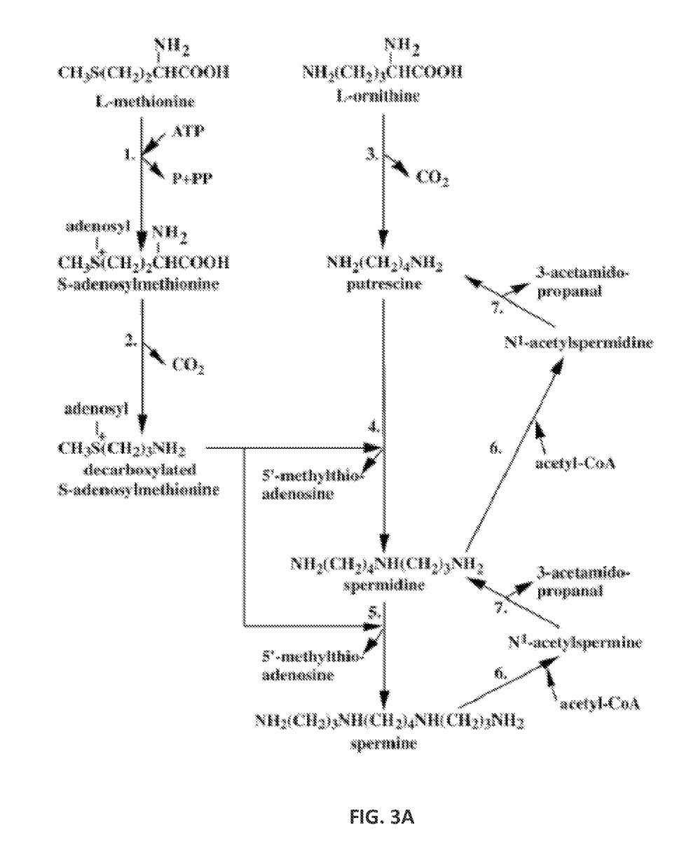

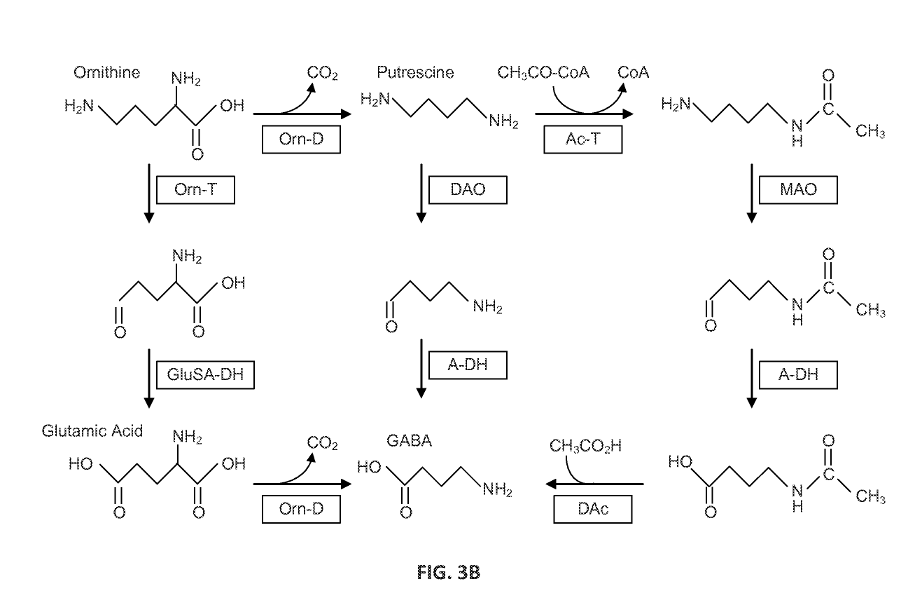

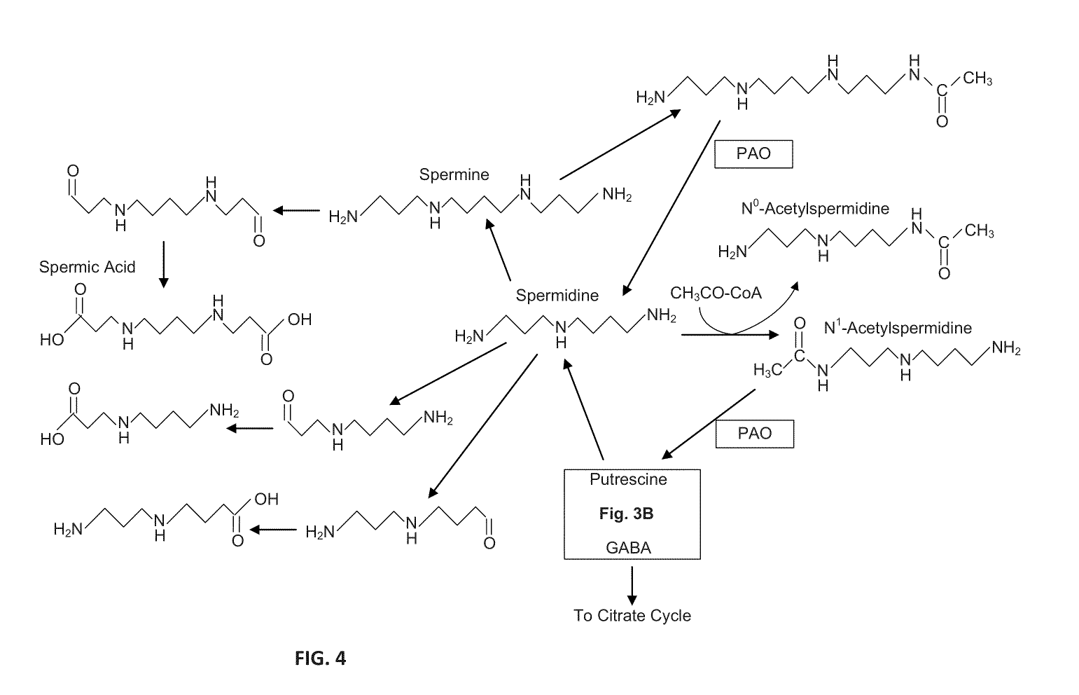

FIG. 3A shows the biosynthesis and interconversion of a variety of polyamines. While these polyamines have been found in both neurons and glial cells, in both the immature and adult central nervous systems, the main cellular locus of ornithine decarboxylase (ODC, enzyme 3 in FIG. 3A) is the neuron. Moreover, during strong functional activation (e g , kindling, epileptic seizures, or neural transplantation), astrocytes and other non-neuronal cells also express ODC and other polyamine-metabolizing enzymes. A membrane potential-dependent transport mechanism for polyamines is found in synaptosomes and glial cells and a proton gradient-dependent transport mechanism for polyamines is found in synaptic vesicles. Transport of polyamines by synaptosomes is blocked by putrescine, agmatine, histidine, and histamine; these compounds, along with norepinephrine, interfere with glial transport. However, in synaptic vesicles, transport of polyamines by synaptosomes is susceptible only to putrescine and histamine blockage.

The most direct substrate for polyamine synthesis is L-ornithine. The process is regulated by two enzymes, ODC (half-life of 30 min) and S-adenosylmethionine decarboxylase (SAMDC) (half-life 30-60 min) (FIG. 3A). Both ODC and SAMDC require pyridoxal 5′-phosphate as a co-factor for activation. These enzymes' activity is rapidly and transiently upregulated by certain pathological “stimuli,” such as epileptic seizures, among others. ODC and SAMDC are downregulated by polyamines through the synthesis of antizyme protein.

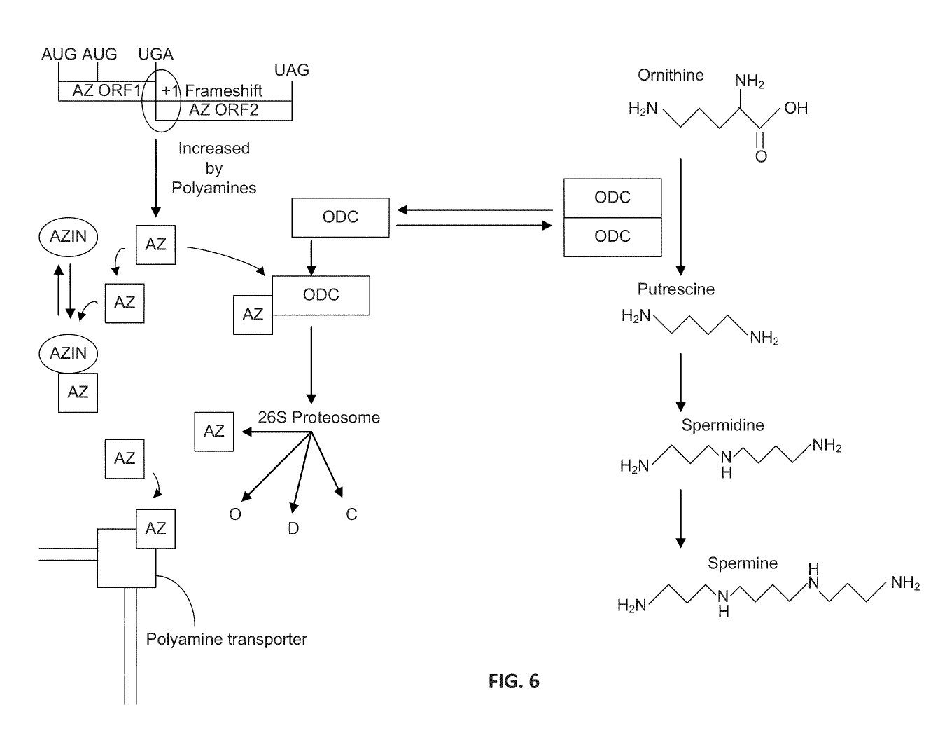

FIG. 6 shows in greater detail the role of antizyme (AZ) and ODC in polyamine metabolism. The ODC dimer catalyzes the production of putrescine, which is then converted into the higher polyamines (e.g., spermidine and spermine). Antizyme is synthesized via a +1 frameshift in translation of the mRNA fusing ORF1 and ORF2 in a manner stimulated by polyamines. Antizyme (AZ) can bind to ODC bringing about degradation by the 26 S proteasome or to antizyme inhibitor (AZIN).

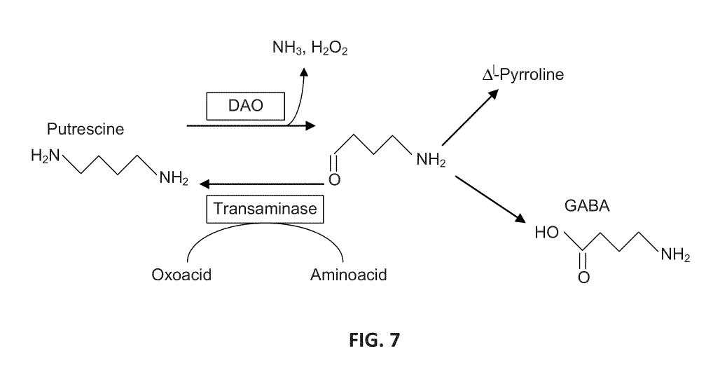

Spermine and spermidine are catabolized through the action of spermidine/spermine N¹-acetyltransferase (SSAT), to yield putrescine, or by oxidation (via serum amine oxidase or intracellular FAD-dependent polyamine oxidase) to produce γ-aminobutyric acid (GABA) and cytotoxic compounds such as hydrogen peroxide, ammonia, and aminoaldehydes. FIG. 3B shows pathways of GABA formation. Polyamine deamination is an alternate path to glutamate decarboxylation for GABA synthesis. On this alternate path, putrescine is a direct precursor. Specifically, removal of one of the two amino groups of putrescine by DAO yields 4-aminobutyraldehyde, which on further oxidation by A-DH yields GABA (FIG. 3B and FIG. 7).

GABA may be also indirectly synthesized from ornithine, not only through the putrescine path mentioned immediately above, but also via δ-transamination of ornithine to glutamic semialdehyde by Orn-T, which upon oxidation by GluSA-DH forms glutamic acid which, when acted upon by its decarboxylase, Glu-D, yields GABA.

FIG. 4 also shows biosynthetic and catabolic reactions of polyamines, with the same enzyme legend as FIG. 3B.

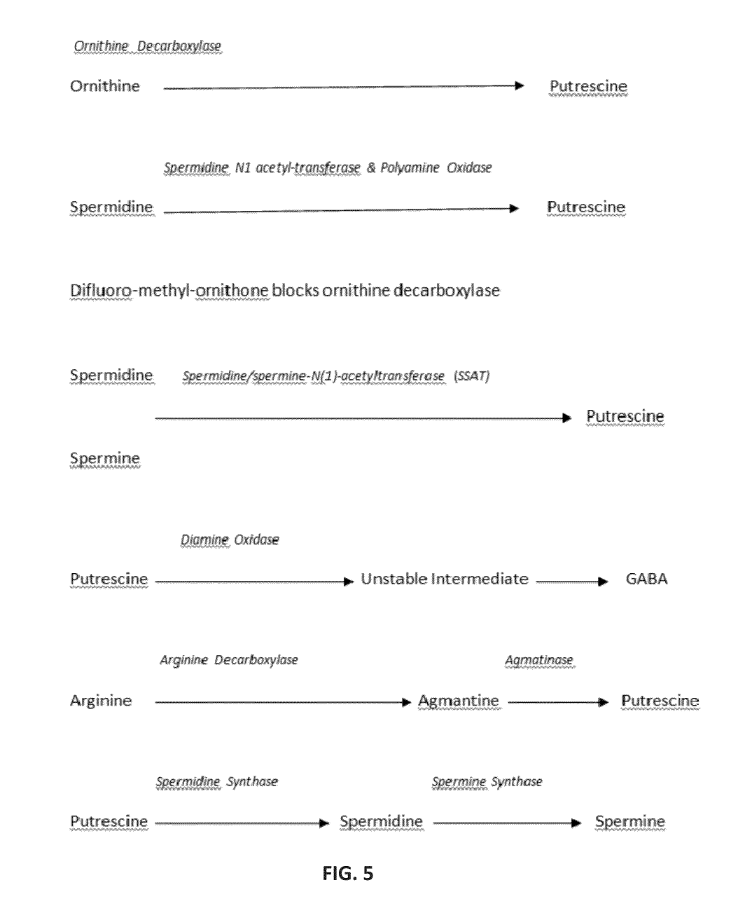

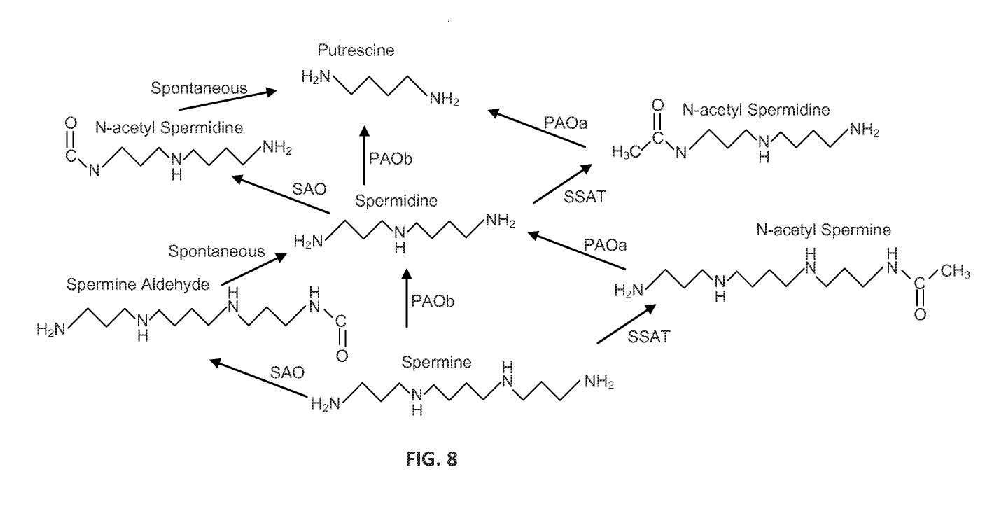

FIG. 5 and FIG. 8 each recapitulate the pathways of biosynthesis and interconversion of polyamines. FIG. 8 additionally indicates which reactions are spontaneous (open arrows) or enzyme driven (filled arrows).

Seizures promote synthesis, recycling, or inter-conversion of putrescine. Since this compound is oxidized to GABA, tracking (e.g., in real-time or in quasi-real time) the ratio of putrescine/GABA concentrations provides information about their rate of synthesis and catabolism. Specifically, an increase in GABA concentration without a concomitant decrease in putrescine/GABA concentration indicates an increase in putrescine synthesis or a decrease in its degradation. Using various statistical tools such as autoregression modeling (e.g., ARMA, ARIMA, FARIMA, etc.), Bayesian models, or Kalman filtering, among others, predictions about trends in changes in their concentrations may be made. Such predictions may be based on the results of assays of these two compounds, of one only (e.g., GABA) or of multiple signals (e.g., electrical, ionic or metabolic). These predictions may be used to automatically trigger one or more actions to restore their concentrations to values associated with a low or lower probability of seizure occurrence. Additionally, a uni-variate or multi-variate probabilistic model may be developed for prevention of seizures, and/or abatement of their intensity, duration, or extent of spread, using information on the state of: a) polyamine synthesis, stores, transport and degradation as well as of their precursors and enzymes; b) intensity, duration and extent of spatial spread of epileptiform/epileptic electrical activity; c) tissue responsiveness to endogenously mediated stimuli (e.g., sensory stimuli, visual stimuli, memory tasks); d) tissue responsiveness to exogenous electrical, chemical, mechanical or thermal stimuli; e) global (patient is asleep) and/or local (high extracellular K⁺) conditions; f) environmental factors (exposure to flickering lights); and/or g) the history of the patient's seizures (e.g. severity of last seizures, time elapsed since last seizure, probability distribution function of seizure energy or inter-seizure intervals), among other factors.

If an elevation in putrescine and/or GABA concentrations is desired to prevent and/or abate a seizure, this elevation may be effected in a number of ways: 1) by increasing the supply of ornithine; 2) by increasing the activity of the enzyme ornithine decarboxylase, which can be done with certain hormones, guanosine triphosphate (GTP), certain amino acids, or insulin; 3) by inflicting a short-lived hypotonic shock to epileptogenic tissue; 5) by inhibiting ornithine transaminase with 5-fluoromethylornithineine; 6) by applying a stimuli (e.g., electrical or thermal currents) to a target tissue; 7) by applying drugs (e.g., chlorpromazine, imipramine, β-adrenergic agonists or a- adrenergic antagonists (e.g., phentolamine) or 8) by GABA-T inhibition, to increase elevation of brain GABA levels and subsequent brain putrescine levels. A decrease of S-adenosylmethionine decarboxylase activity associated with GABA elevation by GABA-T inhibition is followed by an increase in ornithine decarboxylase activity.

Measurements of spermine and/or spermidine, both neuro-active compounds, may be also performed to: a) determine nervous tissue excitability and its “proximity” to a critical threshold, or probability of an inter-ictal-to-ictal transition; b) institute measures to decrease the probability. Measurements of spermine and/or spermidine may be performed with or without simultaneous measurements of putrescine and/or GABA.

Endogenous polyamines, in particular spermine, have been found to block or modulate various ion channel and receptors. Intracellular spermine intrinsically gates and activates inward rectifier K+ channels by directly plugging the ion channel pore. These K+ channels control the resting membrane potential in excitable cells and through it, those cells' firing threshold. Intracellular spermine also causes inward rectification at some subtypes of Ca2+-permeable glutamate receptors by interacting directly with the receptor channel pore. Extracellular spermine has multiple effects at the N-methyl-d-aspartate (NMDA) subtype of glutamate receptor, including stimulation that increases the size of NMDA receptor currents, and voltage-dependent blockage.

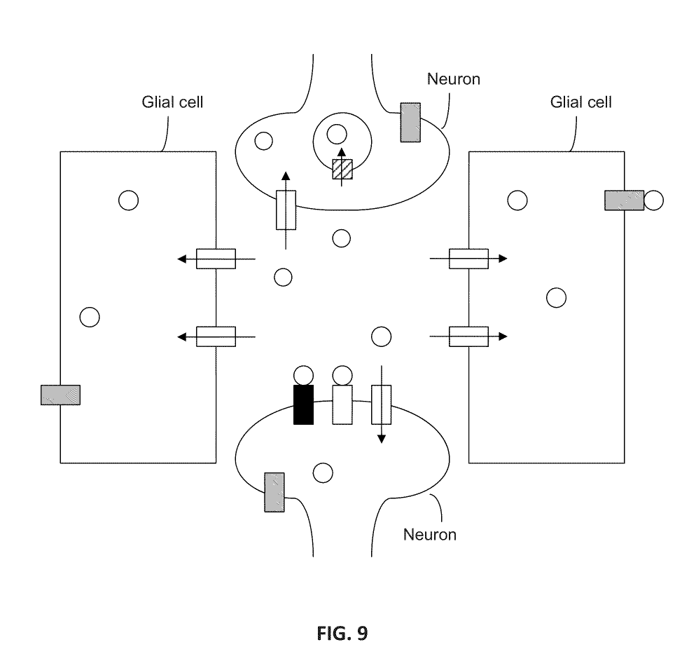

More generally, FIG. 9 shows a model of polyamine circulation with specific reference to the synapse. Polyamine transporters expressed in synaptic vesicles, neuronal cells, and glial cells each have different properties. Polyamines, especially spermine, are accumulated in synaptic vesicles and are released by depolarization. Intracellular polyamines cause blockage and rectification of Kir channels and some subtypes of AMPA/kainate receptors. Extracellular polyamines may modulate glutamate receptors, such as NMDA receptors.

Further continuing the discussion of polyamines and glutamate receptors, a number of polyamine-conjugated arthropod toxins and synthetic polyamine analogues are potent antagonists of glutamate receptors, and may be used to modulate or control epileptogenic or pro-epileptogenic tissue. (Herein, “pro-epileptogenic tissue” refers to that which facilitates seizure emergence and/or occurrence, but from where seizures do not necessarily originate).

The direct actions of polyamines on both ionic transport across membranes and neurotransmitter receptor activity may be exploited for controlling the generation or abatement of seizures. For example, potassium inward rectifying currents (Kir) may be modified in response to increases in intrinsic neuronal excitability or to the presence of seizure precipitants or triggers by changing tissue pH (e.g., alkalinization), either alone on as part of a multifactorial approach involving managing polyamine concentrations, and their precursors and/or enzymes.

Some embodiments of the present disclosure may: 1. anticipate transitions from the interictal to the ictal state by measuring the concentration, rate, and/or spatial extent of changes of precursors, enzymes, end products, and/or metabolites of polyamines such as putrescine or spermidine; 2. maintain a concentration of polyamines within a value range associated with low probability of occurrence of epileptiform activity and/or seizures through endogenous means (e.g., increasing the concentration of the substrates for polyamine synthesis and/or the activity of ornithine decarboxylase, enzyme that catalyzes its synthesis; decreasing polyamine degradation); and/or 3. maintain a concentration of polyamines within a value range associated with low probability of occurrence of epileptiform activity and/or seizures through exogenous means (e.g., delivery of electrical currents at frequencies that stimulate the synthesis of polyamines but do not trigger seizures).

In some embodiments, one or more of these activities may be performed in real time.

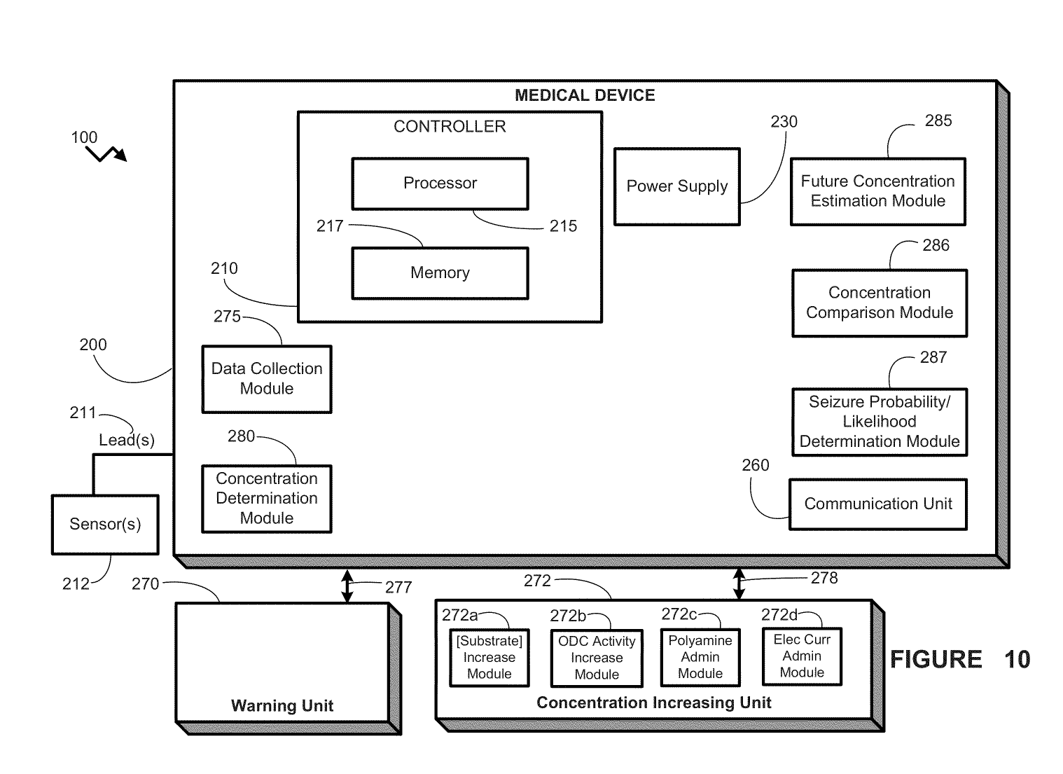

Turning now to FIG. 10, a block diagram depiction of a medical device system 100 comprising a medical device 200 is provided, in accordance with one illustrative embodiment of the present disclosure. In some embodiments, the medical device 200 and/or other components may be implantable in a patient's body, while in other embodiments the medical device 200 and other components may be completely or partially external to the body of the patient.

The medical device 200 may comprise a controller 210 capable of controlling various aspects of the operation of the medical device 200. The controller 210 may be capable of receiving internal data or external data, and in one embodiment, may be capable of causing a polyamine concentration unit 272 to manage (e.g., increase, decrease or maintain a concentration of one or more of putrescine, spermidine, spermine, or GABA, their substrates, precursors, metabolites and/or enzymes) in one or more tissues and/or locations in a patient's nervous system. Generally, the controller 210 may be capable of affecting substantially all functions of the medical device 200.

The controller 210 may comprise various components, such as a processor 215, a memory 217, etc. The processor 215 may comprise one or more microcontrollers, microprocessors, etc., capable of performing various executions of software components. The memory 217 may comprise various memory portions where a number of types of data (e.g., internal data, external data instructions, software codes, status data, diagnostic data, etc.) may be stored. The memory 217 may comprise one or more of random access memory (RAM), dynamic random access memory (DRAM), electrically erasable programmable read-only memory (EEPROM), flash memory, etc.

The medical device 200 may also comprise a power supply 230. The power supply 230 may comprise a battery, voltage regulators, capacitors, etc., to provide power for the operation of the medical device 200, including delivering the therapeutic electrical signal. The power supply 230 comprises a power source that in some embodiments may be rechargeable. In other embodiments, a non-rechargeable power source may be used. The power supply 230 provides power for the operation of the medical device 200, including electronic operations and the electrical signal generation and delivery functions. The power supply 230 may comprise a lithium/thionyl chloride cell or a lithium/carbon monofluoride (LiCFx) cell if the medical device 200 is implantable, or may comprise conventional watch or 9V batteries for external (i.e., non-implantable) embodiments. Other battery types known in the art of medical devices may also be used.

The medical device 200 may also comprise a communication unit 260 capable of facilitating communications between the medical device 200 and various devices. In particular, the communication unit 260 may be capable of providing transmission and reception of electronic signals to and from a polyamine concentration unit 272. The electronic signals may be transmitted and received wirelessly or by cable. The communication unit 260 may include hardware, software, firmware, or any combination thereof

The medical device 200 may also comprise one or more sensor(s) 212 coupled via sensor lead(s) 211 to the medical device 200. The sensor(s) 212 are capable of receiving signals related to a concentration of one or more of putrescine, spermidine, spermine, or GABA, a substrate for the synthesis thereof, of an enzyme for the synthesis thereof, and delivering the signals to the medical device 200. Typically, the sensor(s) 212 may be implanted, although in certain embodiments, the sensor(s) 212 may be external structures be placed on the patient's skin, such as on the patient's head.

In one embodiment, the medical device 200 may comprise a data collection module 275 that is capable of collecting data relating to concentrations of putrescine, spermidine, spermine, or GABA, substrates or enzymes for their synthesis, or one or more of their metabolites. The data collection module 275 may also process or condition such data. The data may be provided by the sensor(s) 212. The data collection module 275 may be capable of performing any necessary or suitable amplifying, filtering, and performing analog-to-digital (A/D) conversions to prepare the signals for downstream processing. In one embodiment, the data collection module 275 may comprise software module(s) that are capable of performing various interface functions, filtering functions, etc. In another embodiment, the data collection module 275 may comprise hardware circuitry that is capable of performing these functions. In yet another embodiment, the data collection module 275 may comprise hardware, firmware, software and/or any combination thereof.

The data collection module 275 may be capable of providing the collected data to one or more of a concentration determination unit 280, a future concentration estimation module 285, a concentration comparison module 286, or a seizure probability/likelihood determination module 287. These various modules will now be discussed in turn.

The concentration determination unit 280 may be capable of determining the concentration of one or more of putrescine, spermidine, spermine, or GABA, substrates or enzymes for their synthesis, or activity level of one or more enzymes, or one or more of their metabolites, in a nervous system tissue and/or location of interest based upon data provided by data collection module 275. The concentration determination unit 280 may implement one or more algorithms to do so. The concentration determination unit 280 may comprise hardware, firmware, software, and/or any combination thereof. The concentration determination unit 280 may be configured to store determined concentration(s) in memory 217 and/or provide determined concentration(s) to one or more of a future concentration estimation module 285, a concentration comparison module 286, or a seizure probability/likelihood determination module 287.

The future concentration estimation module 285 may be capable of estimating a concentration of putrescine, spermidine, spermine, or GABA, substrates or enzymes for their synthesis, or one or more of their metabolites, expected to be present in the tissue/location of interest at a later time. The future concentration estimation module 285 may do so by any appropriate technique. For example, the future concentration estimation module 285 may perform a linear extrapolation of previously determined concentrations. Such a linear extrapolation for one or more of putrescine, spermidine, spermine, or GABA, or substrates or enzymes for the synthesis thereof may be modified based on an extrapolated concentration of another of those compounds. For example, if the concentrations of two compounds are in equilibrium during normal activity of one of the enzymes shown in FIGS. 3-8, an extrapolated increase in the concentration of one compound would be expected, all else being equal, to increase the concentration of the other compound. Alternatively or in addition, the future concentration estimation module 285 may estimate a concentration by one or more other techniques. The future concentration estimation module 285 may comprise hardware, firmware, software, and/or any combination thereof.

The concentration comparison module 286 may be configured to compare (a) first concentration(s) of one or more of putrescine, spermidine, spermine, or GABA, or substrates or enzymes for their synthesis, or one or more of their metabolites, with (a) second, previously-determined concentration(s) of the same or a different compound. For example, the concentration comparison module 286 may be configured to compare a first ratio of e.g., a putrescine concentration and a GABA concentration to a second, previously-determined ratio of a putrescine concentration and a GABA concentration. The concentration comparison module 286 may comprise hardware, firmware, software, and/or any combination thereof

The seizure probability/likelihood determination module 287 may be configured to determine a probability of occurrence of a seizure based on data provided by one or more of the concentration determination module 280, the future concentration estimation module 285, or the concentration comparison module 286. As described above, the concentration(s) of one or more of putrescine, spermidine, spermine, or GABA, substrates or enzymes for their synthesis, or one or more of their metabolites, in a particular nervous system tissue and/or location at one or more times may be indicative of a greater or lesser probability of a seizure occurrence within a given time window. The seizure probability/likelihood determination module 287 may comprise hardware, firmware, software, and/or any combination thereof.

In some embodiments, the seizure probability/likelihood determination module 287 may be configured to determine a seizure susceptibility index relating to a likelihood of an occurrence of a seizure. A “seizure susceptibility index” or “SI” is used herein as a semi-quantitative or qualitative measure or a clinical judgment of a patient's likelihood of suffering a seizure within a predetermined time period. The SI may be based on one or more of the present or an expected future concentration of a polyamine or a substrate or enzyme for synthesis thereof; or one or more patient body signals, such as brain electrical signals; or a certain state (e.g., sleep, during which the likelihood of seizures is higher than during wakefulness); among others.

In some embodiments, based at least in part on one or more outputs of the concentration determination module 280, the future concentration estimation module 285, the concentration comparison module 286, or the seizure probability/likelihood determination module 287, one or more other components of the medical device system 100 may be configured to perform additional actions. These components may receive such outputs via communication unit 260. Although depicted as being external to the medical device 200, in other embodiments (not shown), these components may be internal to a medical device.

In some embodiments, the processor 215 may be configured to (a) determine the concentration of the polyamine, the substrate for synthesis of the polyamine, or the enzyme for synthesis of the polyamine from the collected data; (b) perform one or more of: (b-1) estimate a future concentration of the polyamine, a substrate for synthesis of the polyamine, an enzyme for synthesis of the polyamine, or a metabolite of the polyamine; (b-2) compare the concentration to a reference value; or (b-3) determine, as function of the concentration, a probability of an occurrence of at least one seizure during a first time window; and (c) issue a signal relating to a further action, based on at least one of the determined concentration, the estimated future concentration, the compared concentration, or the determined probability. In other words, in some embodiments, the processor 215 may perform the functions ascribed to the concentration determination module 280, the future concentration estimation module 285, the concentration comparison module 286, and the seizure probability/likelihood determination module 287.

In addition to components of the medical device 200 described above, a medical device system may comprise a warning unit 270. The warning unit 270 may be configured to warn at least one of a patient, a caregiver, or a healthcare provider of (a) particular concentration(s), (an) estimated concentration(s), or (a) compared concentration(s) of one or more of putrescine, spermidine, spermine, or GABA, substrates or enzymes for their synthesis, or one or more of their metabolites, at one more nervous system locations and/or times, or warn of a determined seizure probability. Such a warning may comprise a sound, a light, an electronic message, or other types of warnings apparent to the person of ordinary skill in the art having the benefit of the present disclosure.

In one embodiment, the medical device system 100 may comprise a polyamine concentration unit 272. The polyamine concentration unit 272 may be configured to increase a concentration of one or more of putrescine, spermidine, spermine, or GABA, or substrates or enzymes for the synthesis thereof, in one or more tissues and/or locations of the nervous system of the patient. Doing so may result in a reduction of the likelihood or probability of occurrence of a seizure, and/or abatement of such a seizure as may occur. Generally, higher concentrations of a polyamine are correlated with lower probabilities of a seizure. (The person of ordinary skill in the art will understand that very high concentrations of the polyamine may have detrimental effects on a patient and should be avoided). To do so, the polyamine concentration unit 272 may comprise one or more modules 272a-d.

Substrate concentration increase module 272a may be configured to increase a concentration of one or more substrates of an enzyme depicted in FIGS. 3-8 as catalyzing the conversion of the substrate(s) to a compound for which concentration increase is desired.

ODC activity increase module 272b may be configured to increase ODC activity in the desired tissue/location. As shown in, e.g., FIG. 3, by doing so, putrescine concentrations may be increased, and from putrescine, GABA, spermidine, and/or spermine concentrations may also be increased.

Polyamine administration module 272c may be configured to increase the concentration of one or more of putrescine, spermidine, spermine, or GABA, or substrates or enzymes for the synthesis thereof, by administering that compound or those compounds to the desired tissue/location.

Electrical current administration unit 272d may be configured to increase the concentration of one or more of putrescine, spermidine, spermine, or GABA, or substrates or enzymes for the synthesis thereof, by delivering an electrical current to the desired tissue/location.

More information about how modules 272a-d may be implemented is set forth supra.

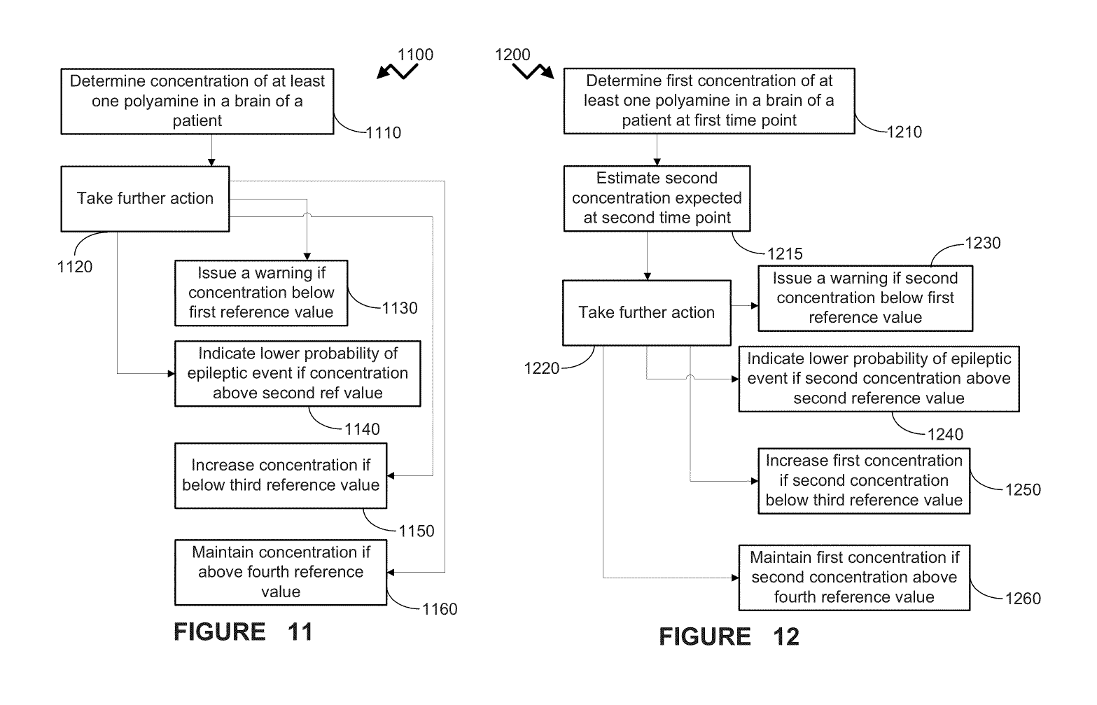

Turning to FIG. 11, a flowchart of a method 1100 in accordance with one embodiment of the present disclosure is presented. A concentration of at least one polyamine selected from putrescine, spermidine, spermine, or γ-aminobutyric acid (GABA) is determined at 1110 in a brain of a patient.

The concentration may be determined at 1110 substantially continuously; on a repeating schedule; on demand by a healthcare provider; in response to a change in a body signal of the patient, such as a brain electrical activity; in response to at least one of time of day, state of the epileptogenic network, state of the brain, state of the patient, or in response to changes in the patient's environment; or two or more thereof, among others.

Thereafter, the method 1100 may comprise taking at 1120 a further action selected from issuing a warning at 1130 if the concentration is below a first reference value; indicating a lower probability of an epileptic event at 1140 if the concentration is above a second reference value; increasing the concentration at 1150 if the concentration is below a third reference value; or maintaining the concentration at 1160 above a fourth reference value.

As should be apparent, in some embodiments, two or more of issuing at 1130, indicating at 1140, increasing at 1150, or maintaining at 1160 may be performed. Also, any of the first through fourth reference values may be the same as one or more of the other reference values, or all four reference values may be different.

In some embodiments, increasing the concentration of the polyamine at 1150 may comprise activating an endogenous mechanism. In some embodiments, the endogenous mechanism may be selected from increasing the concentration of at least one substrate for polyamine synthesis, increasing the concentration or activity of ornithine decarboxylase, increasing a concentration of a cofactor of ornithine decarboxylase, or decreasing the degradation of one or more of the polyamines. Alternatively or in addition, increasing the concentration of the polyamine at 1150 may comprise administering at least one of the polyamine to the patient, or an electrical current to a neural tissue of the patient. In some embodiments, the neural tissue may be selected from neural tissue is selected from a vagus nerve, a cranial nerve other than a vagus nerve, a peripheral nerve, a spinal root, a portion of a spinal cord, or a portion of a brain.

The determining at 1110, issuing at 1130, indicating at 1140, increasing at 1150, or maintaining at 1160 may be performed by any apparatus and/or technique described supra.

FIG. 12 presents a flowchart of a method 1200 in accordance with one embodiment of the present disclosure. In method 1200, a first concentration of at least one of: a polyamine selected from putrescine, spermidine, spermine, or γ-aminobutyric acid (GABA); a substrate for synthesis of putrescine, spermidine, spermine, or GABA; an enzyme for synthesis of putrescine, spermidine, spermine, or GABA; or a metabolite of putrescine, spermidine, spermine, or GABA is determined at 1210 at a first time point in a brain of a patient. Thereafter, the method 1200 may further comprise estimating at 1215 a second concentration, expected at a second, future time point, of the polyamine or related compounds. By “future time point” is meant a time point not yet reached as of the time the first concentration is determined.

Subsequently, the method 1200 may further comprise taking a further action at 1220 selected from issuing a warning at 1230 if the second concentration is below a first reference value; indicating a lower probability of an epileptic event at 1240 if the second concentration is above a second reference value; increasing the first concentration at 1250 if the second concentration is below a third reference value; or maintaining the first concentration above a fourth reference value at 1260.

In some embodiments, the increasing at 1250 may comprise activating an endogenous mechanism selected from increasing the concentration of at least one substrate for polyamine synthesis or increasing the activity of ornithine decarboxylase. Alternatively or in addition, the increasing at 1250 may comprise administering at least one of the polyamines to the patient, an electrical current to a neural tissue of the patient, or heating or cooling neural tissue of the patient, such as to one or more of the neural tissues described supra.

The determining at 1210, estimating at 1215, issuing at 1230, indicating at 1240, increasing at 1250, or maintaining at 1260 may be performed by any apparatus and/or technique described supra.

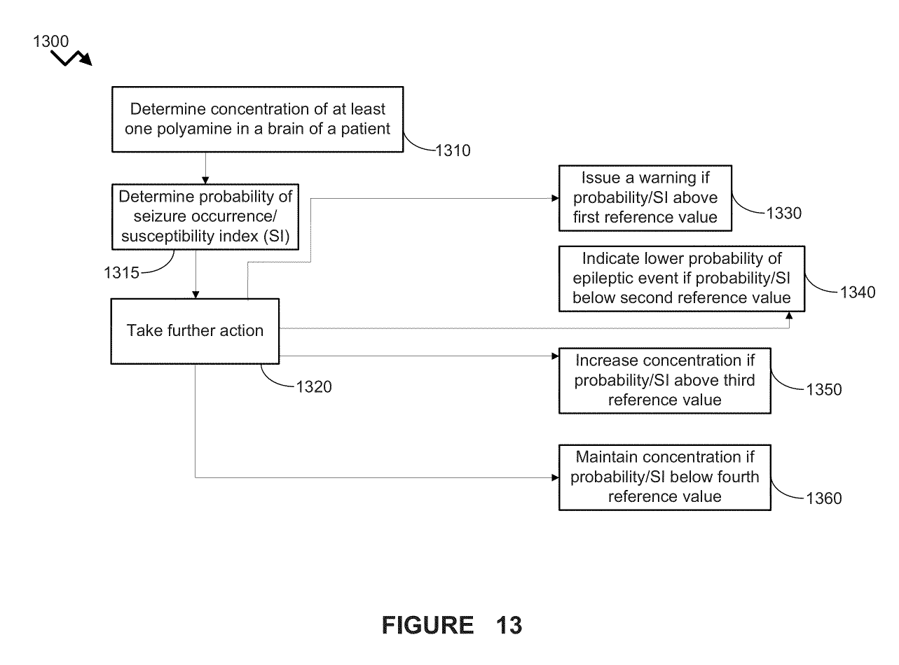

Turning to FIG. 13, a flowchart of a method 1300 in accordance with one embodiment of the present disclosure is depicted. In method 1300, a concentration of at least one polyamine selected from putrescine, spermidine, spermine, or γ-aminobutyric acid (GABA) is determined at 1310 in a brain of a patient. Based on the concentration, the method 1300 may comprise determining at 1315 a probability of an occurrence of at least one seizure during a first time window. Alternatively or in addition, instead of a probability of a seizure occurrence, the method 1300 may comprise determining at 1315 a seizure susceptibility index (SI), or a likelihood of a seizure during the first time window. Thereafter, the method 1300 may comprise taking at 1320 a further action selected from issuing at 1330 a warning if the probability or SI is above a first reference value; indicating at 1340 a lower probability of an epileptic event if the probability or SI is below a second reference value; increasing at 1350 the concentration if the probability or SI is above a third reference value; or maintaining at 1360 the concentration if the probability of SI is below a fourth reference value.

In some embodiments, the increasing at 1350 may comprise activating an endogenous mechanism selected from increasing the concentration of at least one substrate for polyamine synthesis, increasing the activity of ornithine decarboxylase, increasing a concentration of a cofactor of ornithine decarboxylase, or decreasing the degradation of one or more of the polyamines. Alternatively or in addition, the increasing at 1350 may comprise administering at least one of the polyamine to the patient, or an electrical current to a neural tissue of the patient.

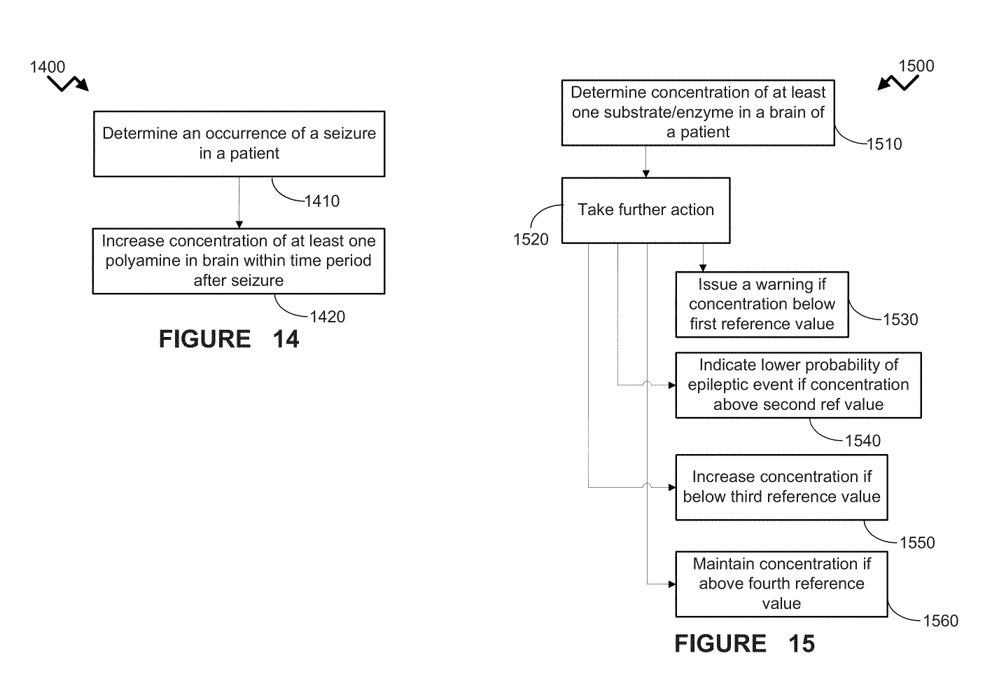

Turning to FIG. 14, a flowchart of a method 1400 according to some embodiments of the present disclosure is depicted. The method 1400 may comprise determining at 1410 an occurrence of a seizure in a patient. Determining at 1410 may be based on clinical observation, electrographic observation (e.g., EEG or ECoG data), or various uni- or multi-modal observations, e.g., cardiac data, other autonomic data, kinetic data, responsiveness or awareness data, or other neurological data, among others, such as described by other patents or patent applications assigned to Cyberonics, Inc., or Flint Hills Scientific, LLC.

The method 1400 may also comprise increasing at 1420 a concentration of at least one polyamine selected from putrescine, spermidine, spermine, or γ-aminobutyric acid (GABA) in a brain of the patient in response to the determining of the occurrence of the seizure and within a predetermined time period thereafter. Increasing at 1420 may reduce the likelihood or the probability of a next seizure occurring within the transient period of increased susceptibility following a seizure (e.g., a particular forward-looking time window), and/or reduce a next seizure's severity. In some embodiments, the predetermined time period after the occurrence of the seizure may be from 1 sec to 24 hr, such as from about 5 min to about 30 min.

Increasing the concentration at 1420 may involve an endogenous mechanism or another mechanism, such as those described above with reference to steps 1150, 1250, and 1250.

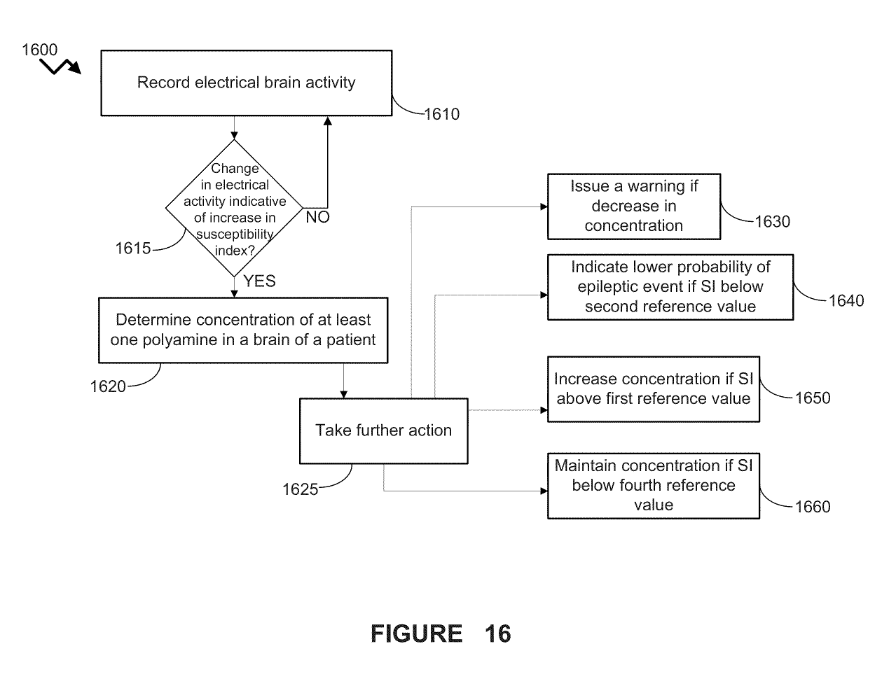

Turning to FIG. 15, a method 1500 is depicted. Many of the steps of method 1500 are similar to those of method 1100, with the significant difference that method 1500 comprises determining at 1510 a concentration of a substrate for synthesis of putrescine, spermidine, spermine, or γ-aminobutyric acid (GABA), or a concentration or level of activity of an enzyme for synthesis of putrescine, spermidine, spermine, or GABA, in a brain of a patient. Thereafter, a warning may be issued at 1530 or a lower probability of an epileptic event may be indicated at 1540 based on the concentration of the substrate or enzyme. Similarly, the concentration of the substrate or enzyme may be increased at 1550 or maintained at 1560.

Turning to FIG. 16, a method 1600 is depicted. The method 1600 may comprise recording at 1610 electrical brain activity of a patient. Recording at 1610 may be substantially continuous, periodic, performed on the demand of a healthcare provider, or performed automatically in response to an input such as those described above, among others. The electrical brain activity may comprise epileptiform discharges, seizures, or a DC potential. (“Epileptiform discharges” may also be termed “spikes”).

If it is determined at 1615 the electrical brain activity has not changed at all, or has not changed in a manner indicative of an increase in the patient's susceptibility index (SI) or probability of seizure occurrence, then further recording at 1610 may be performed. On the other hand, if it is determined at 1615 that the electrical brain activity has changed in a manner indicative of an increase in the patient's susceptibility index (SI) or probability, then a concentration of at least one polyamine selected from putrescine, spermidine, spermine, or γ-aminobutyric acid (GABA) in a brain of a patient may be determined at 1620. Electrical brain activity that may indicate an increase in the patient's SI or probability may include the change in electrical activity is at least one of a change in a spectral property, a change in morphology, a change in amplitude, a change in rate of occurrence, a change in duration of epileptiform discharges, a change in duration of bursts of epileptiform discharges, a change in duration of seizures, a change in the number or amplitude of slow waves associated with epileptiform discharges, a change in the phase relation between a spike and a slow wave, a change in the distribution of inter-epileptiform discharges or inter-seizure intervals, or a change in the distribution of epileptiform discharges.

Thereafter, the method 1600 may comprise taking at 1625 a further action selected from issuing at 1630 a warning if the change in electrical activity is associated with a decrease in concentration of the polyamine; initiating at least one action at 1650 to increase the concentration in response to the seizure susceptibility index or probability being above a first reference value; indicating at 1640 a lower probability or likelihood of an epileptic event if the probability or susceptibility index are below a second reference value; or maintaining at 1660 the concentration above a third reference value.

Any of the methods described above may further comprise the delivery of a therapy to the patient. The therapy and/or parameters thereof may be based at least in part on one or more of a polyamine concentration, a polyamine precursor concentration, a polyamine synthesis enzyme concentration, an enzyme activity level, an enzyme co-factor concentration, a polyamine metabolite concentration, a seizure probability, or an SI value. In some embodiments, the therapy may be an electrical stimulation of the vagus nerve, such as that commercially available from Cyberonics, Inc.

The methods depicted in FIGS. 11-16 and described above may be governed by instructions that are stored in a non-transitory computer readable storage medium and that are executed by, e.g., a processor 217 of the medical device 200. Each of the operations shown in FIGS. 11-16 may correspond to instructions stored in a non-transitory computer memory or computer readable storage medium. In various embodiments, the non-transitory computer readable storage medium includes a magnetic or optical disk storage device, solid state storage devices such as flash memory, or other non-volatile memory device or devices. The computer readable instructions stored on the non-transitory computer readable storage medium may be in source code, assembly language code, object code, or other instruction format that is interpreted and/or executable by one or more processors.

The numbered paragraphs immediately below refer to various embodiments of the present disclosure.

1. The present disclosure may relate to a system (or method or apparatus) to:

Determine in vivo the concentration of putrescine in brain tissue (epileptogenic and pro-epileptogenic);

Compare said concentration to a reference value;

Issue a warning if said concentration is at or below the fiftieth percentile of reference values or one standard deviation below the mean; and

Institute measures to increase concentration of putrescine in said brain tissues.

2. The present disclosure may relate to a system (or method or apparatus) to

Determine the concentration of putrescine in epileptogenic or pro-epileptogenic brain tissue;

Determine the concentration of substrates for putrescine and/or of the enzymes involved in its synthesis;

Determine the rate of synthesis and catabolism of putrescine;

Using these data determine if the concentration of putrescine will increase, decrease or remain constant over a certain time period; and

Institute measures to prevent said decrease and issue a warning, if the concentration of putrescine will decrease.

3. The present disclosure may relate to a system (or method or apparatus) to:

Determine the probability of seizure occurrence as a function of the concentration of putrescine in epileptogenic and pro-epileptogenic tissue;

Issue a warning if said probability is near or at an unsafe value for the type of activity the patient is performing or will perform; and

Institute measures to lower said probability to safe value for the type of activity the patient is performing or will perform and cancel the warning if this is accomplished.

101. A non-transitory computer readable program storage unit encoded with instructions that, when executed by a computer, perform a method, comprising:

determining, at a first time point, a first concentration or activity level of at least one of: a polyamine selected from putrescine, spermidine, spermine, or γ-aminobutyric acid (GABA); a substrate for synthesis of putrescine, spermidine, spermine, or GABA; a metabolite of putrescine, spermidine, spermine, or GABA; or an enzyme for synthesis of putrescine, spermidine, spermine, or GABA; in a brain of a patient;

estimating a second concentration or activity level of said at least one polyamine, substrate, metabolite, or enzyme, expected at a second, future time point; and

taking a further action selected from:

issuing a warning if said second concentration or activity level is expected to be below a first reference value;

indicating a lower probability or likelihood of an epileptic event if said second concentration or activity level is expected to be above a second reference value;

increasing said first concentration if said second concentration or activity level is expected to be below a third reference value; or

maintaining said first concentration or activity level above a fourth reference value.

102. The non-transitory computer readable program storage unit of numbered paragraph 101, encoded with instructions that, when executed by a computer, perform the method of numbered paragraph 101, wherein said at least one measure comprises activating an endogenous mechanism selected from increasing the concentration of at least one substrate for polyamine synthesis, increasing the activity of ornithine decarboxylase, increasing a concentration of a cofactor of ornithine decarboxylase, or decreasing the degradation of one or more of said polyamines.

102. The non-transitory computer readable program storage unit of numbered paragraph 101, encoded with instructions that, when executed by a computer, perform the method of numbered paragraph 101, wherein said increasing said concentration or activity level of said polyamine comprises administering at least one of said polyamine to said patient, or an electrical current to a neural tissue of said patient.

201. A non-transitory computer readable program storage unit encoded with instructions that, when executed by a computer, perform a method, comprising:

determining a concentration or activity level of at least one polyamine selected from putrescine, spermidine, spermine, or γ-aminobutyric acid (GABA) in a brain of a patient;

determining a probability or likelihood of an occurrence of at least one seizure during a first time window, based on said concentration; and

taking a further action selected from:

issuing a warning if said probability or likelihood is above a first reference value;

indicating a lower probability or likelihood of an epileptic event if said probability is below a second reference value;

increasing said concentration or activity level if said probability or likelihood is above a third reference value; or

maintaining said concentration or activity level if said probability or likelihood is below a fourth reference value.

202. The non-transitory computer readable program storage unit of numbered paragraph 201, encoded with instructions that, when executed by a computer, perform the method of numbered paragraph 201, wherein said at least one measure comprises activating an endogenous mechanism selected from increasing the concentration of at least one substrate for polyamine synthesis, increasing the activity of ornithine decarboxylase, increasing a concentration of a cofactor of ornithine decarboxylase, or decreasing the degradation of one or more of said polyamines.

203. The non-transitory computer readable program storage unit of numbered paragraph 201, encoded with instructions that, when executed by a computer, perform the method of numbered paragraph 201, wherein said increasing said concentration or activity level of said polyamine comprises administering at least one of said polyamine to said patient, or an electrical current to a neural tissue of said patient.

301. A non-transitory computer readable program storage unit encoded with instructions that, when executed by a computer, perform a method, comprising:

determining a concentration of a substrate for synthesis of putrescine, spermidine, spermine, or γ-aminobutyric acid (GABA), or a concentration or activity level of an enzyme for synthesis of putrescine, spermidine, spermine, or GABA, in a brain of a patient; and

taking a further action selected from:

issuing a warning if said concentration is below a first reference value;

indicating a lower probability or likelihood of an epileptic event if said concentration or activity level is above a second reference value;

increasing said concentration or an activity level of said enzyme if said concentration or activity level is below a third reference value; or

maintaining said concentration above a fourth reference value.

302. The non-transitory computer readable program storage unit of numbered paragraph 301, encoded with instructions that, when executed by a computer, perform the method of numbered paragraph 301, wherein said increasing said concentration or said activity level of said substrate or enzyme comprises activating an endogenous mechanism selected from increasing a concentration of a cofactor of said enzyme, or decreasing the degradation of said substrate or said enzyme.

303. The non-transitory computer readable program storage unit of numbered paragraph 301, encoded with instructions that, when executed by a computer, perform the method of numbered paragraph 301, wherein said increasing said concentration or said activity level of said substrate or enzyme comprises administering an electrical current to a neural tissue of said patient.

304. The non-transitory computer readable program storage unit of numbered paragraph 303, encoded with instructions that, when executed by a computer, perform the method of numbered paragraph 303, wherein said neural tissue is selected from a vagus nerve, a cranial nerve other than a vagus nerve, a peripheral nerve, a spinal root, a portion of a spinal cord, or a portion of a brain.

401. A medical device system, comprising:

(i) at least one sensor configured to collect data relating to at least one of: a concentration or activity level of a polyamine selected from putrescine, spermidine, spermine, or γ-aminobutyric acid (GABA) or a metabolite thereof; a concentration or activity level of a substrate for synthesis of putrescine, spermidine, spermine, or GABA; or a concentration or activity level or an activity level of an enzyme for synthesis of putrescine, spermidine, spermine, or GABA; in a brain of a patient;

(ii) at least one processor configured to:

determine said concentration or activity level of said polyamine, said concentration or activity level of said substrate for synthesis of said polyamine, said concentration or said activity level of said enzyme for synthesis of said polyamine, or said concentration or activity level of said metabolite of said polyamine;

determine a seizure susceptibility index relating to a probability or likelihood of an occurrence of a seizure;

perform at least one of:

issue a warning if said susceptibility index is above a first reference value;

indicate a lower probability or likelihood of an epileptic event if said susceptibility index is below a second reference value;

increase said concentration or activity level if said susceptibility index is above a third reference value; or

maintain said concentration or activity level if said susceptibility index is below a fourth reference value.

402. The medical device system of numbered paragraph 401, wherein said processor is configured to increase said concentration or activity level by at least one of: increasing the concentration of at least one substrate for polyamine synthesis, increasing the activity of ornithine decarboxylase, increasing a concentration of a cofactor of ornithine decarboxylase, or decreasing the degradation of one or more of said polyamines.

403. The medical device system of numbered paragraph 401, said processor is configured to increase said concentration or activity level by administering at least one of said polyamine to said patient, or an electrical current to a neural tissue of said patient.

501. A non-transitory computer readable program storage unit encoded with instructions that, when executed by a computer, perform a method, comprising:

recording electrical brain activity,

determining a concentration of a polyamine selected from putrescine, spermidine, spermine, or γ-aminobutyric acid (GABA) or a metabolite thereof; a concentration of a substrate for synthesis of putrescine, spermidine, spermine, or GABA; or a concentration/activity level of an enzyme for synthesis of putrescine, spermidine, spermine, or GABA in a brain of a patient in response to a change in said electrical activity, wherein said change is indicative of an increase in a seizure susceptibility index, and

taking a further action selected from:

issuing a warning if said change in electrical activity is associated with a decrease in concentration of said polyamine, or

initiating at least one action to increase said concentration in response to said susceptibility index being above a first reference value;

indicating a lower probability or likelihood of an epileptic event if said susceptibility index is below a second reference value;

maintaining said concentration above a third reference value.

502. The non-transitory computer readable program storage unit of numbered paragraph 501, encoded with instructions that, when executed by a computer, perform the method of numbered paragraph 501, wherein said brain electrical activity comprises epileptiform discharges, seizures, or a DC potential.

503. The non-transitory computer readable program storage unit of numbered paragraph 501, encoded with instructions that, when executed by a computer, perform the method of numbered paragraph 501, wherein said change in electrical activity is at least one of a change in a spectral property, a change in morphology, a change in amplitude, a change in rate of occurrence, a change in duration of epileptiform discharges, a change in duration of bursts of epileptiform discharges, a change in duration of seizures, a change in the number or amplitude of slow waves associated with epileptiform discharges, a change in the distribution of inter-epileptiform discharges or inter-seizure intervals, or a change in the distribution of epileptiform discharges.

601. A non-transitory computer readable program storage unit encoded with instructions that, when executed by a computer, perform a method, comprising:

determining a concentration of at least one polyamine selected from putrescine, spermidine, spermine, or γ-aminobutyric acid (GABA), or a metabolite, substrate, or enzyme for synthesis thereof, or an activity level of said enzyme, in a brain of a patient in response to at least one of time of day, state of the epileptogenic network, state of the brain, state of the patient, or in response to changes in the environment, and

taking a further action selected from:

issuing a warning if said concentration is below a first reference value,

indicating a lower probability or likelihood of an epileptic event if said concentration is above a second reference value;

increasing said concentration if said concentration is below a third reference value; or

maintaining said concentration above a fourth reference value.

602. The non-transitory computer readable program storage unit of numbered paragraph 601, encoded with instructions that, when executed by a computer, perform the method of numbered paragraph 601, wherein said increasing said concentration of said polyamine comprises activating an endogenous mechanism selected from increasing the concentration of at least one substrate for polyamine synthesis, increasing the activity of ornithine decarboxylase, increasing a concentration of a cofactor of ornithine decarboxylase, or decreasing the degradation of one or more of said polyamines.

603. The non-transitory computer readable program storage unit of numbered paragraph 601, encoded with instructions that, when executed by a computer, perform the method of numbered paragraph 601, wherein said increasing said concentration of said polyamine comprises administering at least one of said polyamine to said patient, or an electrical current to a neural tissue of said patient.

701. A non-transitory computer readable program storage unit encoded with instructions that, when executed by a computer, perform a method, comprising:

recording electrical brain activity in a brain of a patient,

determining an epileptiform activity in said brain of said patient based on said recorded electrical activity, and

taking a further action selected from:

issuing a warning of said epileptiform activity, or

initiating at least one action to increase a concentration or activity level of a polyamine, a substrate for synthesis thereof, or an enzyme for synthesis thereof, in response to said epileptiform activity;

indicating a lower probability or likelihood of an epileptic event if said epileptiform activity is below a reference value;

or maintaining said concentration or activity level of said polyamine, said substrate, or said enzyme above a third reference value.

What is claimed:

1. A non-transitory computer readable program storage unit encoded with instructions that, when executed by a computer, perform a method, comprising:
determining at least one of (i) a concentration of at least one polyamine selected from putrescine, spermidine, spermine, or γ-aminobutyric acid (GABA) in a brain of a patient, (ii) a concentration of a substrate for synthesis of putrescine, spermidine, spermine, or GABA in said brain of said patient, (iii) a concentration or an activity level of an enzyme for synthesis of putrescine, spermidine, spermine, or GABA in said brain of said patient, or (iv) a concentration of a metabolite of one of putrescine, spermidine, spermine, or GABA in said brain of said patient; and
taking a further action selected from:
issuing a warning if said concentration or activity level is below a first reference value;
indicating a lower probability or likelihood of an epileptic event if said concentration or activity level is above a second reference value;
increasing said concentration if said concentration or activity level is below a third reference value; or
maintaining said concentration or activity level above a fourth reference value.

2. The non-transitory computer readable program storage unit of claim 1, encoded with instructions that, when executed by a computer, perform the method of claim 1, wherein said increasing said concentration of said polyamine comprises activating an endogenous mechanism selected from increasing the concentration of at least one substrate for polyamine synthesis, increasing the activity of ornithine decarboxylase, increasing a concentration of a cofactor of ornithine decarboxylase, or decreasing the degradation of one or more of said polyamines.

3. The non-transitory computer readable program storage unit of claim 1, encoded with instructions that, when executed by a computer, perform the method of claim 1, wherein said increasing said concentration of said polyamine comprises administering at least one of said polyamine to said patient, or an electrical current to a neural tissue of said patient.

4. The non-transitory computer readable program storage unit of claim 3, encoded with instructions that, when executed by a computer, perform the method of claim 3, wherein said neural tissue is selected from a vagus nerve, a cranial nerve other than a vagus nerve, a peripheral nerve, a spinal root, a portion of a spinal cord, or a portion of a brain.

5. A non-transitory computer readable program storage unit encoded with instructions that, when executed by a computer, perform a method, comprising:
determining an occurrence of a seizure in a patient;
increasing a concentration of at least one polyamine selected from putrescine, spermidine, spermine, or γ-aminobutyric acid (GABA) in a brain of said patient, a concentration of a substrate for synthesis of putrescine, spermidine, spermine, or GABA in said brain, or a concentration or an activity level of an enzyme for synthesis of putrescine, spermidine, spermine, or GABA in said brain in response to said determining, within a predetermined time period after the occurrence of said seizure.

6. The non-transitory computer readable program storage unit of claim 5, encoded with instructions that, when executed by a computer, perform the method of claim 5, wherein said increasing said concentration of said polyamine comprises activating an endogenous mechanism selected from increasing the concentration of at least one substrate for polyamine synthesis, increasing the activity of ornithine decarboxylase, increasing a concentration of a cofactor of ornithine decarboxylase, or decreasing the degradation of one or more of said polyamines.

7. The non-transitory computer readable program storage unit of claim 5, encoded with instructions that, when executed by a computer, perform the method of claim 5, wherein said increasing said concentration of said polyamine comprises administering at least one of said polyamine to said patient, or an electrical current to a neural tissue of said patient.

8. The non-transitory computer readable program storage unit of claim 1, encoded with instructions that, when executed by a computer, perform the method of claim 1, wherein the method further comprises determining a probability or likelihood of an occurrence of at least one seizure during a first time window, based on said concentration; and
taking a second further action selected from:
issuing a warning if said probability or likelihood is above a fifth reference value;
indicating a lower probability or likelihood of an epileptic event if said probability or likelihood is below a sixth reference value;
increasing said concentration if said probability or likelihood is above a seventh reference value; or
maintaining said concentration if said probability or likelihood is below an eighth reference value.

9. The non-transitory computer readable program storage unit of claim 1, encoded with instructions that, when executed by a computer, perform the method of claim 1, wherein said determining is in response to at least one of a time of day, a state of the epileptogenic network, a state of the brain, a state of the patient, or at least one change in the environment.

10. A medical device system, comprising:
(i) at least one sensor configured to collect data relating to at least one of: a concentration of a polyamine selected from putrescine, spermidine, spermine, or γ-aminobutyric acid (GABA); a concentration of a substrate for synthesis of putrescine, spermidine, spermine, or GABA; a concentration of a metabolite of putrescine, spermidine, spermine, or GABA; or a concentration or an activity level of an enzyme for synthesis of putrescine, spermidine, spermine, or GABA; in a brain of a patient;
(ii) at least one processor configured to:
(ii-a) determine said concentration or said activity level of said polyamine, said substrate, said metabolite, or said enzyme from said collected data;
(ii-b) perform one or more of:
(ii-b-1) estimate a future concentration or activity level of at least one polyamine, said substrate, said metabolite, or said enzyme;
(ii-b-2) compare said concentration or activity level to a reference value; or
(ii-b-3) determine, as function of said concentration or activity level, a probability or likelihood of an occurrence of at least one seizure during a first time window; and
(ii-c) issue a signal relating to a further action, based on at least one of said estimated future concentration or activity level, said compared concentration or activity level, or said determined probability or likelihood; and
at least one of:
(iii) a warning unit configured to provide a warning relating to at least one of said estimated future concentration or activity level, said compared concentration or activity level, or said determined probability or likelihood;
(iv) a concentration increasing unit configured to increase said concentration or activity level, based on at least one of said estimated future concentration or activity level, said compared concentration or activity level, or said determined probability or likelihood.
(v) a probability indication unit configured to indicate a lower probability of an epileptic event, based on at least one of said estimated future concentration or activity level, said compared concentration or activity level, or said determined probability or likelihood; or
(vi) a concentration maintaining unit configured to maintain said concentration or activity level, based on at least one of said estimated future concentration or activity level, said compared concentration or activity level, or said determined probability or likelihood.

11. The medical device system of claim 10, wherein said concentration increasing unit is configured to perform at least one of: (iv-a) increase the concentration of at least one substrate for polyamine synthesis, (iv-b) increase the activity of ornithine decarboxylase, (iv-c) increase a concentration of a cofactor of ornithine decarboxylase, or (iv-d) decrease the degradation of one or more of said polyamines.

12. The medical device system of claim 10, wherein said concentration increasing unit is configured to: (iv-c) administer at least one of (iv-c-1) said polyamine to said patient, or (iv-c-2) an electrical current to a neural tissue of said patient.
